(19) United States
(12) Patent Application Publication
(10) Pub. No.: US 2011/0230396 A1
(43) Pub. Date: Sep. 22, 2011

(54) ANTI-INFECTIVE COMPOUNDS AND USES THEREOF

(75) Inventor: Ola Winqvist, Svankarrsvagen (SE)

(73) Assignee: ITH Immune Therapy Holdings AB, Stockholm (SE)

(21) Appl. No.: 13/059,997

(22) PCT Filed: Aug. 20, 2009
(86) PCT No.: PCT/SE2009/000386

§ 371 Date: May 26, 2011

(30) Foreign Application Priority Data
Aug. 21, 2008 (SE) 0801816-0

(51) Int. Cl. A61K 38/17; A61P 31/10

(52) U.S. Cl. 514/3.4; 514/3.3

(57) ABSTRACT

A medicament to prevent or cure fungal infections of the skin or body openings in an immunologically compromised person or animal can comprise Cystatin S, Cystatin SA, Cystatin SN, or similar agents.

### FIELD OF THE INVENTION

The invention relates to compounds which have a prophylactic and/or curative effect on infections of skin and body openings. More particularly, the invention relates to anti-fungal properties of Cystatins.

### BACKGROUND OF THE INVENTION

Cystatins are a superfamily of lysosomal enzyme-inhibiting proteins characterized by a single chain of about 115 to 122 amino acids with a molecular weight of about 13,000 Daltons, having two disulfide bonds. Their action inhibits cysteine proteases which action contributes to a healthy state in humans.

Innumerate infections can plague humans, some having nearly insignificant effects and others being fatal. The infectious agents behind these diseases are equally large in number; some classes of infectious agents include fungi, bacteria, and viruses. Often, organisms which are neutral or even beneficial to an organism can become a detriment if their population reaches a critical density or their location on or within the host changes.

Furthermore, various disease states exist which contribute to a host's ability to prevent and/or fight infection. This includes both genetic disease states and acquired disease states.

One example of a genetic disease state is Autoimmune Polyendocrine Syndrome type I (APS I), in which mutations in the autoimmune regulator gene (AIRE) can result in immunological destruction of endocrine organs. An example of an acquired disease state is Acquired Immune Deficiency Syndrome (AIDS); there a patient's immune system is attacked by a virus which results in depleted immune capabilities. In both cases, the patient has a decreased capacity to resist or fight infection. But it is not only diseased individuals who suffer infection. Otherwise healthy individuals become ill from time to time. Infants and the elderly are especially prone to infection, as are patients undergoing strenuous medical treatment such as chemotherapy or organ transplant recipients. In response to this, practitioners are continually looking to improve the arsenal of treatments available to fight infection. In modern Western medical practice, pharmaceuticals are the most-often relied upon means to prevent or treat infection. Decades of research and refinement allow treating physicians to choose from general, broad-spectrum agents to specific targeted agents against a particular infectious agent, or even against specific strains of agents.

One drawback of pharmaceutical treatment can be side effects, which are present to some degree in all medicaments. So while eradication or prevention of infection is desirable, ways to minimise the negative consequences of treatment is an ongoing challenge to workers in the art.

Another drawback of pharmaceutical treatment is the risk of the microorganism developing resistance to the treatment.

As but one brief example, anti-fungals such as imidazoles and triaazoles can be very effective but their side effects can include altered drug metabolism in the liver and blocking of steroid synthesis. Thus, despite the range of fungicidal products presently available there remains a need in the art to provide both broad spectrum and targeted treatments to prevent and reduce fungal infection.

### OBJECTS OF THE INVENTION

It is a primary object of the invention to provide a method and a means for treating fungal infection in a person or animal, in particular in an immunologically compromised person or animal.

It is another object of the invention to provide a method and a means for treating fungal infection in a person or animal in which the causative agent has developed resistance to an antifungal drug.

Still another object of the invention is to provide a method and a means for treating fungal infection in a manner that does not risk eliciting an adverse immune response in the so treated person or animal.

A further object of the invention is to provide a method and a means for treating fungal infection in a person or animal the metabolic burden on the recipient of which is reduced in respect of that by conventional antifungal drugs.

Still further objects of the invention will be evident from the following summary of the invention, a number of figures in an attached drawing, the description of preferred embodiments, and the appended claims.

### SUMMARY OF THE INVENTION

The present invention relates to an anti-infectious treatment, in particular an anti-fungal treatment, with prophylactic and curative effects.

In one embodiment of the invention, Cystatin S is used for the preparation of a medicament for preventing or treating fungal infection of skin or body openings. The medicament could comprise about 5-500 μM Cystatin S, such as from about 50-100 μM Cystatin S. The Cystatin S can comprise Cystatin SA 1.

The medicament of this embodiment can further comprise one or more emulsifiers, carriers, solvents, pH adjusters, preservatives, sweeteners and flavourants, such as emulsifying wax, isopropyl myristate, glycerine, lactic acid, sodium hydroxide, sorbic acid, mineral oil, white petrolatum, benzoic acid, butylhydroxyanisol, oleomacroglycerides, pegaxol 7 stearate, paraffin, and water. The medicament can be used for preventing or treating infection of skin or body openings in a patient having at least one mutation in the autoimmune regulator gene, such as autoimmune polyendocrine syndrome type I.

According to this embodiment, the medicament is particularly useful in preventing or treating fungal infection attributable to Candida albicans.

In another embodiment of the invention, Cystatin S is used for the preparation of a medicament for preventing or treating fungal infections of the skin or body openings.

In yet another embodiment of the invention, a method of preventing or reducing an infection of skin or a body opening in a person or animal infected by a fungus is provided, which method comprises administering a pharmaceutically effective amount of at least one Cystatin S to the patient. The medicament could comprise about 5-500 μM Cystatin S, such as from about 50-100 μM Cystatin S. The Cystatin S can comprise Cystatin SA 1.

The medicament used in the method of this embodiment can further comprise one or more emulsifiers, carriers, solvents, pH adjusters, preservatives, sweeteners and flavourants, such as emulsifying wax, isopropyl myristate, glycerine, lactic acid, sodium hydroxide, sorbic acid, mineral oil, white petrolatum, benzoic acid, butylhydroxyanisol, oleomacroglycerides, pegaxol 7 stearate, paraffin, and water. The medicament can be used for preventing or treating infection of skin or body openings in a person or animal whose production of Cystatin S is genetically impaired or impaired by an acquired disease, in particular a person or animal having at least one mutation in the autoimmune regulator gene, such as autoimmune polyendocrine syndrome type I or suffering from AIDS or being in a nutritionally deficient state.

According to this embodiment, the method can comprise administering a medicament to prevent or treat fungal infection attributable to Candida albicans.

According to a preferred aspect of the invention, infection is by a fungus having developed resistance to one or more fungicidal drugs, the method comprising substituting said one or more fungicidal drug by Cystatin S, in particular Cystatin S comprising Cystatin SA 1. In particular, the fungus is one having developed resistance to a fungicidal drug selected from polyene antifungal, imidazole antifungal, triazole antifungal. Polyene antifungal drugs capable of eliciting resistance comprise amphotericin, nystatin, natamycin or candicin. Imidazole antifungal drugs capable of soliciting resistance comprise ketoconazole, miconazole, crotrimazole, econazole, oxiconazole, sertaconazole or tioconazole. Triazole antifungal drugs capable of soliciting resistance comprise fluoconazole, itraconazole, posaconazole or voriconazole.

As used herein “person or animal” includes all relevant organisms and is not intended to be limiting. The term includes not only non-human mammals such as dogs but other animals such as chicken and fish. The invention is applicable generally to all organisms in which it has effect and may be employed for any reason, such as improving quality of life, improving overall health, and economic benefit, to name but a few.

As used herein, “body opening” refers to both natural body openings of a person or animal and their proximal structures. Examples include nasal passages, oral cavities, ears, eyes, urethra, vagina, and rectum.

As used herein, “Cystatin” refers to any member of the cystatin protein family, such as A, B, C, D, E/M, F, G, H-kg, L-kg, S, SA, and SN, including full-length proteins and active fragments thereof. Cystatin may refer to a mixture of different cystatins or a single cystatin. Where applicable, the discussion of Cystatins encompasses sequences encoding the same. A thorough description of the cystatin superfamily and its interrelationships can be found in, e.g., Abrahamsson et al. Cystatins, Biochem. Soc. Symp. 70, 179-199 (2003), which is expressly incorporated by reference herein.

“Cystatin S” refers generally to the class of cystatins that share significant similarity in structure or function with Cystatin S, Cystatin SA, (including both Cystatin SA 1 and Cystatin SA 2) and Cystatin SN. “Cystatin S” includes active fragments of the proteins, see for example D. P. Dickinson, Crit. Rev. Oral Biol. Med 2002. Also encompassed in the term are sequences which encode the same.

Medicaments according to the present invention may include any number of items in addition to the active agent. These are well known in the art and include emulsifiers, carriers, preservatives, flavourants, colourants, pH adjusting agents, coating agents, and dispersing agents.

Forms suitable for dosing are also known to skilled workers, and include liquids, tablets for swallowing, dissolving or chewing, chewing gums, toothpastes and mouth rinses, sprays, gels, films, and suppositories.

Dosing regimens will vary widely depending on the purpose of the use, the particular cystatin or combination of cystatins employed and their purities, the route of administration, the adjuvants or carriers and any other materials present in the medicament. Therefore, it is noted that one way to determine an effective dosage amount is to note the purpose of use and select the route of administration. For example, for an orally-administered preventative anti-fungal one would consider the fungal agents whose proliferation could be prevented through oral administration and experiment on them using different cystatins to develop an understanding the approximate dosage strength and frequency to suppress proliferation. These and other methods of determining dose route and regimen are well known in the art and as such lie well within the grasp of the skilled worker.

### BRIEF DESCRIPTION OF THE FIGURES

In order that the present invention may be clearly understood and readily carried into effect reference will now be made, by way of example, to the accompanying drawings in which:

FIGS. 1A and 1B show electrophoretic analysis for Cystatin SA 1 expression in control and APS I patients, respectively;

FIGS. 2A and 2B show data comparing control and APS I saliva for their ability to inhibit Candida albicans, where the hyphae form is shown in FIG. 2A and the yeast form in 2B; and

FIG. 3 summarises experimental data comparing the ability of Cystatin SA 1 and Nystatin to inhibit Candida albicans.

### DETAILED DESCRIPTION

Cystatin S offers hope in the field of new and improved preventative and curative medicaments. As detailed herein, it has a pronounced effect on infectious agents such as fungi. Since it is a naturally-occurring substance in patients the risk of complications and side-effects is reduced.

For preventative purposes, Cystatin S treatment may be particularly well-suited to patients who do not have sufficient amounts of Cystatin S in the appropriate place in their body. Another preventative use is for patients subjected to unusually high amounts of infectious agents, such as people in the health care profession or animals reared under intense conditions.

According to the present invention one patient population which may benefit from prophylactic administration of Cystatin S are those suffering from APS I. Despite their overly-activated immune system, these patients are unable to resist colonization of the fungus Candida albicans (C. albicans) and develop Chronic Mucucutaneous Candidiasis (CMC) at an early age. CMC infection causes immense distress for patients and can be carcinogenic over the long term.

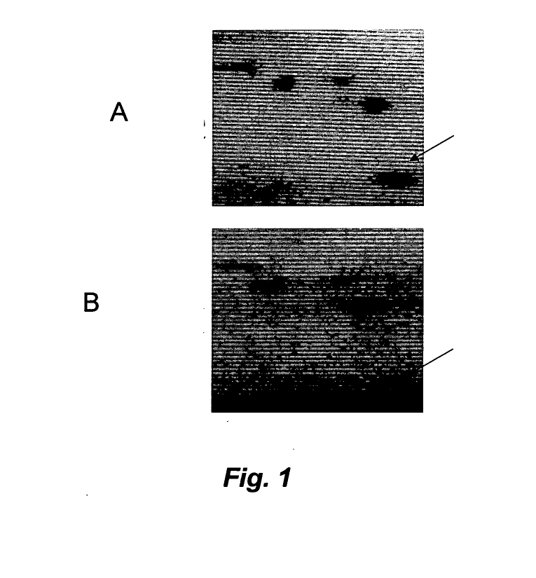

In the pursuit of the present invention it has been found that APS I patients have decreased ability to inhibit Candida growth compared to healthy subjects. By analysing the salivary protein profile of APS I patients with 2D electrophoresis and comparing the same to that of healthy subjects, it was found that APS I patients lack expression of Cystatin SA 1. See FIG. 1, where the presence of Cystatin SA 1 in healthy subjects (FIG. 1A) is seen whereas the same marker is lacking in APS I patients (FIG. 1B). Cystatin SA is typically present in healthy human saliva at approximately 1 μM.

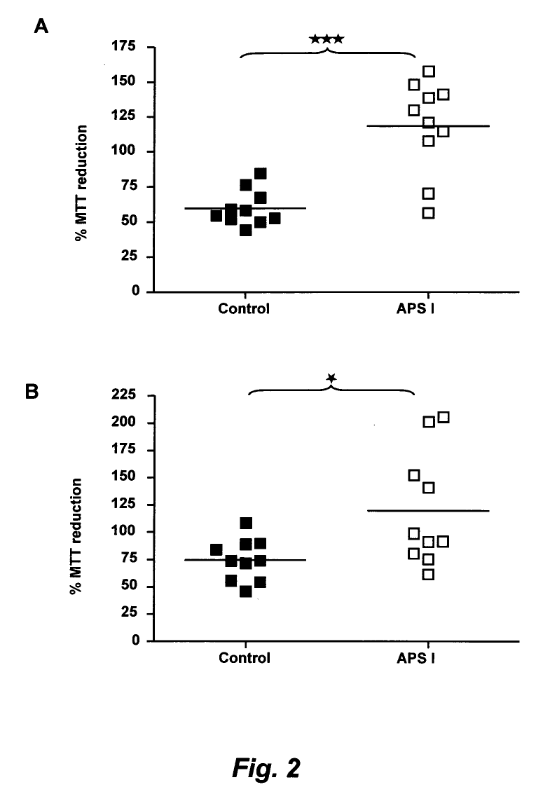

The protein Cystatin SA 1 is encoded by the CST 2 gene located on chromosome 20p11.21. When tested in vitro, saliva having normal amounts of Cystatin SA 1 protein was shown to inhibit both the hyphae and yeast forms of C. albicans. Referring to FIG. 2, saliva from healthy subjects (Control) inhibits C. albicans, whereas saliva from APS I patients (APS I) does not. FIG. 2A shows results when tested against hyphae form and FIG. 2B shows results as relevant for the yeast form.

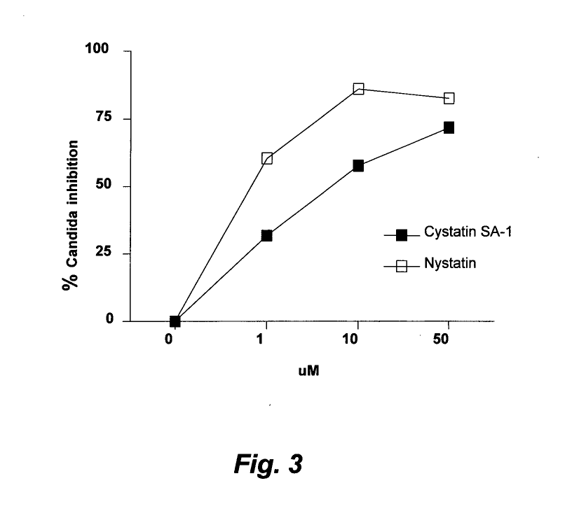

To confirm this surprising find, Cystatin SA 1 inhibition of Candida growth in vitro was evaluated by culturing synthesised Cystatin SA 1 protein with C. Albicans and measuring the growth rate. FIG. 3A summarises the findings which demonstrate the inhibitory effect of Cystatin SA 1 on Candida. For purposes of comparison, the experiment was repeated using a commonly-prescribed anti-fungal, Nystatin, which also inhibits Candida as shown in the figure. Using common treatments, 1 mL of Nystatin might be administered four times daily, burdening in particular the liver and potentially interfering with other bodily systems. Longer-term use of this or other anti-fungals can result in a decrease in the normal fungal flora and expose a patient to attack by drug-resistant fungi.

In contrast, Cystatin S is normally found in healthy patients and can be efficiently metabolised by the patient without any expectation of side effects. This is particularly relevant for patients who are candidates for long-term prophylactic use of Cystatin S such as patients with APS I and AIDS.

In addition to reducing unwanted side-effects for patients, Cystatin S treatment reduces the chance of drug-resistant fungi, bacteria, and viruses by not allowing patients to effectively serve as breeding grounds for drug-resistant strains. This has a benefit for society as a whole, not only for patient groups in particular need of prophylactic treatment.

### Example 1

A patient presenting an acute fungal infection of the mouth is treated orally with 15.0 μM Cystatin SA in a sweetened carrier. The treatment is repeated for 7 days during which time strict oral hygiene is observed. At the conclusion of treatment the infection is cleared and the mouth presents a normal oral flora.

### Example 2

A patient suffering from early stage AIDS and suffering oral discomfort and irregular bowels is presumed to be experiencing side effects of standard anti-viral drug therapy. Nonetheless a combination of Cystatin S, Cystatin SA, and Cystatin SN is prepared and formulated into oral (0.5 μM cystatins, lozenge) and interrectal (3.0 μM cystatins, suppository) dosage forms. The same are administered 2 times daily for one month. Over the course of the treatment the patient's reported condition improves.

### Example 3

A female patient suffering from recurrent urinary tract infections was treated with long term prophylactic antibiotic treatment. This led to occurrence of bacterial resistance. Patients with recurrent urinary tract infections have a genitical predisposition to, for example, recurrent E. Coli infections due to local factors in the urethra promoting bacterial migration. Suitable amounts of Cystatin S, Cystatin SA, and/or Cystatin SN are prepared and formulated into locally applicable formulation. This prevents the entry and colonization of the urethra with bacteria.

### Example 4

Seven different species of birds housed in a zoo enclosure are exposed to regular contact with human and other animal pathogens. On average, there is one mild infection observed per month. Whenever noted, an infected bird is removed to a separate enclosure and strict anti-infectious measures are enforced regarding care and handling of the bird, to prevent spread. This separate handling comes at an increased cost.

As a prophylactic measure the diet provided to the birds is supplemented with Cystatin S at an amount calculated to result in an average salivary Cystatin S concentration of 2 μM. After six months of treatment employee perception of infection rates is decreased, and an extrapolated average of 0.8 infections per month is observed.

Thus, a new way for Cystatin S to defend against the establishment and proliferation of infectious agents in body openings and on the skin has surprisingly been found and described herein.

Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for preventing or treating fungal infection of skin or body openings in a person or animal whose production of Cystatin S is genetically impaired or impaired by an acquired disease, comprising administering a pharmaceutical composition comprising an effective amount of Cystatin S.

2. The method of claim 1, wherein the infection is attributable to Candida albicans.

3. The method of claim 1, wherein the impairment is caused by autoimmune polyendocrine syndrome type I.

4. The method of claim 1, wherein the impairment is caused by at least one mutation in an autoimmune regulator gene.

5. The method of claim 1, wherein the impairment is caused by AIDS.

6. The method of claim 1, wherein the impairment is caused by a nutritional deficiency.

7. The method of claim 1, wherein the Cystatin S comprises Cystatin SA 1.

8. The method of claim 1, wherein the medicament comprises about 5-500 μM Cystatin S, such as from about 50-100 μM Cystatin S.

9. The method of claim 1, wherein the medicament further comprises one or more emulsifiers, carriers, solvents, pH adjusters, preservatives, sweeteners and flavourants, such as emulsifying wax, isopropyl myristate, glycerine, lactic acid, sodium hydroxide, sorbic acid, mineral oil, white petrolatum, benzoic acid, butylhydroxyanisol, oleomacroglycerides, pegaxol 7 stearate, paraffin, and water.

10. A method of preventing or reducing a fungal infection of skin or a body opening in a person or animal whose production of Cystatin S is genetically impaired or impaired by an acquired disease, comprising administering a pharmaceutically effective amount of at least one Cystatin S to the person or animal.

11. The method of claim 10, wherein the medicament comprises about 5-500 μM Cystatin S, such as from about 50-100 μM Cystatin S.

12. The method of claim 10, wherein the Cystatin S comprises Cystatin SA 1.

13. The method of claim 10, wherein the medicament further comprises one or more emulsifiers, carriers, solvents, pH adjusters, preservatives, sweeteners and flavourants, such as emulsifying wax, isopropyl myristate, glycerine, lactic acid, sodium hydroxide, sorbic acid, mineral oil, white petrolatum, benzoic acid, butylhydroxyanisol, oleomacroglycerides, pegaxol 7 stearate, paraffin, and water.

14. The method of claim 10, wherein the impairment is caused by any of at least one mutation in the person or animal's autoimmune regulator gene; autoimmune polyendocrine syndrome type I; AIDS; a nutritional deficiency.

15. The method of claim 10, wherein the infection is attributable to Candida albicans.

16. The method of claim 10, wherein infection is by a fungus having developed resistance to one or more fungicidal drugs, the method comprising substituting said one or more fungicidal drug by Cystatin S, in particular Cystatin S comprising Cystatin SA 1.

17. The method of claim 16, wherein the one or more fungicidal drug is selected from a polyene antifungal, an imidazole antifungal, and a triazole antifungal.

18. The method of claim 16, wherein the one or more fungicidal drug comprises a polyene antifungal, in particular amphotericin, nystatin, natamycin or candicin.

19. The method of claim 16, wherein the one or more fungicidal drug comprises an imidazole, in particular ketoconazole, miconazole, crotrimazole, econazole, oxiconazole, sertaconazole or tioconazole.

20. The method of claim 16, wherein the one or more fungicidal drug comprises a triazole, in particular fluoconazole, itraconazole, posaconazole or voriconazole.
